(19) United States
(12) Patent Application Publication
(10) Pub. No.: US 2006/0041238 A1
(43) Pub. Date: Feb. 23, 2006

(54) Fluid collection system and method

(76) Inventor: Michael L. Bowen, Mansfield, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/197,638

(22) Filed: Aug. 4, 2005

Related U.S. Application Data
(60) Provisional application No. 60/603,740, filed on Aug. 23, 2004.

(51) Int. Cl. A61F 13/15

(52) U.S. Cl. 604/378; 604/313

(57) ABSTRACT

A fluid collection mat and a system for collecting fluids using a vacuum source is disclosed. The fluid collection mat may be formed from multiple layers of material. At least one layer of the material may be formed from absorbent material. Another layer may be used to regulate fluid flow from the absorbent layer. Still another layer of the material distributes suction from a vacuum source through the mat adjacent to the layer regulating fluid flow from the absorbent layer.

### RELATED APPLICATION

This application claims the benefit of U.S. Provisional Application No. 60/603,740, filed Aug. 23, 2004, the contents of which are hereby incorporated by reference in their entirety.

### TECHNICAL FIELD

This invention relates in general to the field of collecting fluids and in particular to fluid collection systems and fluid collection mats.

### BACKGROUND

Many industrial processes and medical procedures produce fluid wastes which must be collected, stored and properly disposed. Frequently, such fluid waste may be hazardous or potentially hazardous. Waste fluids may contain chemicals, bacteria or other organisms which present potential health risks to personnel working at a facility, visitors and/or outside third parties. Waste fluids may also present a contamination hazard to products and/or processes. Increased concern with cleanliness standards and personal protection requirements in many industrial environments, such as semiconductor fabrication and nanotechnology related processes require increased attention to eliminating undesired waste fluids. Accumulations of undesired fluids in any work area may present a safety hazard.

### SUMMARY

In accordance with teachings of the present invention, disadvantages and problems with previous equipment and methods for collecting fluids have been substantially reduced or eliminated. One aspect of the present invention may include forming a fluid collection mat having a base layer or first layer formed from fluid impervious material. A second layer or fluid pervious layer may be disposed upon the base layer. The second layer is preferably formed from very porous material which allows substantially unrestricted fluid flow therethrough. A third layer or fluid flow regulator may be disposed on the second layer opposite from the base layer. The fluid flow regulator may be formed from generally fluid impervious material with a limited number of fluid flow paths formed therein. A fourth layer or absorbent layer may be disposed on the fluid flow regulator opposite from the second layer. A fifth layer or perforated layer may be disposed on the absorbent layer opposite from the fluid flow regulator.

Technical benefits of the present invention include providing a fluid collection pad formed from relatively soft, foldable or bendable materials. As a result, such fluid collection pads may be easily stored when not in use. Also, such fluid collection pads may be satisfactorily used even when placed in irregularly shaped locations.

Further technical benefits of the present invention includes the use of relatively low cost and light weight materials to form a fluid collection mat. A wide variety of woven fabrics, nonwoven fabrics and plastic films may be satisfactorily used to form one or more layers of the fluid collection mat. For many applications disposable materials may be used to form the fluid collection mat.

One embodiment of the present invention may include attaching a suction hose or conduit with only one portion of a fluid collection mat formed in accordance with teachings of the present invention. One or more layers of material used to form the fluid collection mat will preferably regulate fluid flow through the mat to provide relatively uniform distribution of associated low pressure vacuum throughout the mat even though the suction hose or conduit may be coupled with only one portion of the mat. Present invention allows the use of a wide variety of fluid connections and fluid conduits extending between a fluid collection mat formed in accordance with teachings of the present invention and a source of low pressure suction.

### BRIEF DESCRIPTION OF THE DRAWINGS

A more complete and thorough understanding of the present inventions and advantages thereof, reference is now made to the following written description taken in conjunction with the accompanying drawings, in which:

FIG. 1 is a schematic drawing showing one example of a fluid collection system having a fluid collection mat or pad incorporating teachings of the present invention;

FIG. 2 is a schematic drawing showing an exploded isometric view of various layers of material used to form a fluid collection mat or pad in accordance with teachings of the present invention; and

FIG. 3 is a schematic drawing in section with portions broken away showing a fluid collection mat or pad formed in accordance with teachings of the present invention.

### DETAILED DESCRIPTION

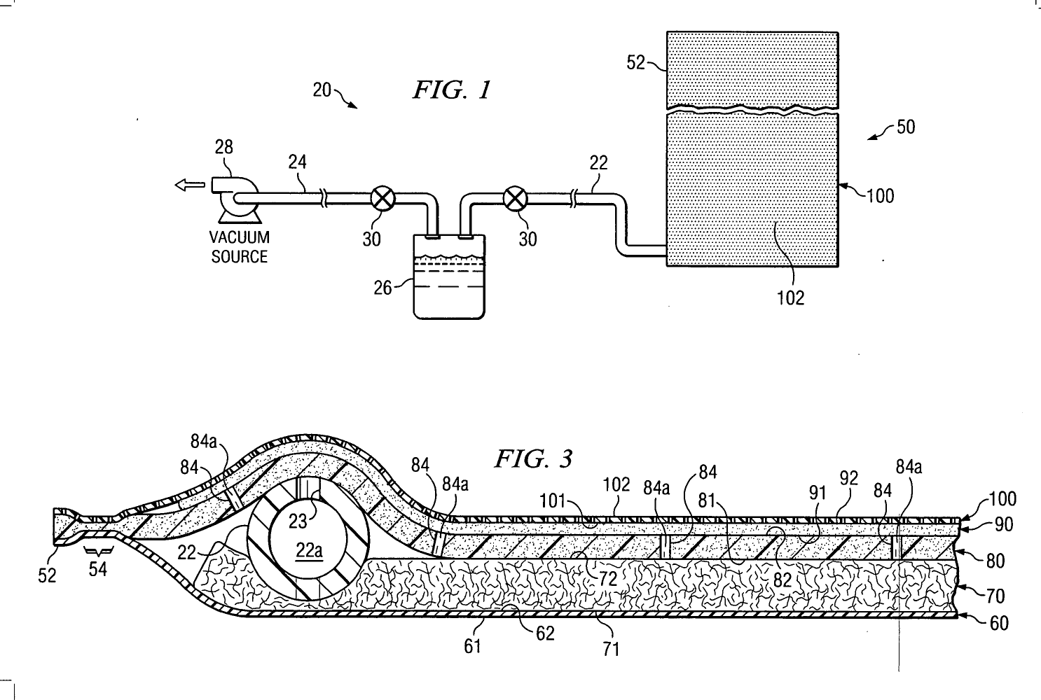

Preferred embodiments of the present invention and its advantages are best understood by reference to FIGS. 1-3 of the drawings, like numerals being used for like and corresponding parts of the various drawings.

The term “fluid” may be used to include any gas, liquid or mixture of gases and liquids. Various types of particulate matter, nanoparticles and/or aerosols may be entrained within such fluids. The term “aerosols” may be used to describe any insoluble liquid or particulate matter in a gaseous suspension.

The term “mat” may be used to include a pad or any other structure having multiple layers of material incorporating teachings of the present invention.

FIGS. 1-3 show one example of a fluid collection system and associated fluid collection mat incorporating teachings of the present invention. For embodiments such as shown in FIG. 1, fluid collection system 20 may include various components such as first conduit 22, second conduit 24, container 26, vacuum source 28 and fluid collection pad 50. Various aspects of the present invention may be described with respect to low pressure suction. However, fluid collection mats incorporating teachings of the present invention may be satisfactorily used with any type of pressure or vacuum (low, moderate, or high). Technical benefits of the present invention include regulating fluid flow within a pad or mat to provide a generally uniform distribution of applied suction or vacuum.

For some applications fluid collection pad 50 may be used in a medical facility such as a surgical operating room or in a trauma center. Disposable materials may be used to form fluid collection pad 50 so that pad 50 may be stored and disposed of using the same procedures and techniques as other disposable products associated medical facilities and medical procedures.

Vacuum source 28 and/or container 26 may be representative of the types of low pressure suction available in such medical facilities. However, a fluid collection pad incorporating teachings of the present invention may be satisfactorily used at a wide variety of work stations and other locations including clean rooms, chemical laboratories and food processing facilities. Also, fluid collection pads incorporating teachings of the present invention may be used during maintenance on fluid systems, particularly fluid systems associated with hazardous materials. For some applications fluid collection pads may be formed from materials which are specifically selected to be compatible with hazardous materials associated with a work station or fluid system.

For some applications conduit 22 may directly couple mat 50 with vacuum source 28. For other applications, one end of conduit 22 may be coupled with mat 50 and the other end coupled with container 26. Container 26 may be particularly useful if hazardous or potentially hazardous fluids flow through conduit 22. Second conduit 24 may be used to couple container 26 with vacuum source 28.

Various types of flow control devices 30 may be included within conduits 22 and 24. Also, filters and fluid separators (not expressly shown) may be included as part of system 20. For some applications multiple mats 50 may be coupled with container 26. Also, multiple containers 26 may be coupled with vacuum source 28.

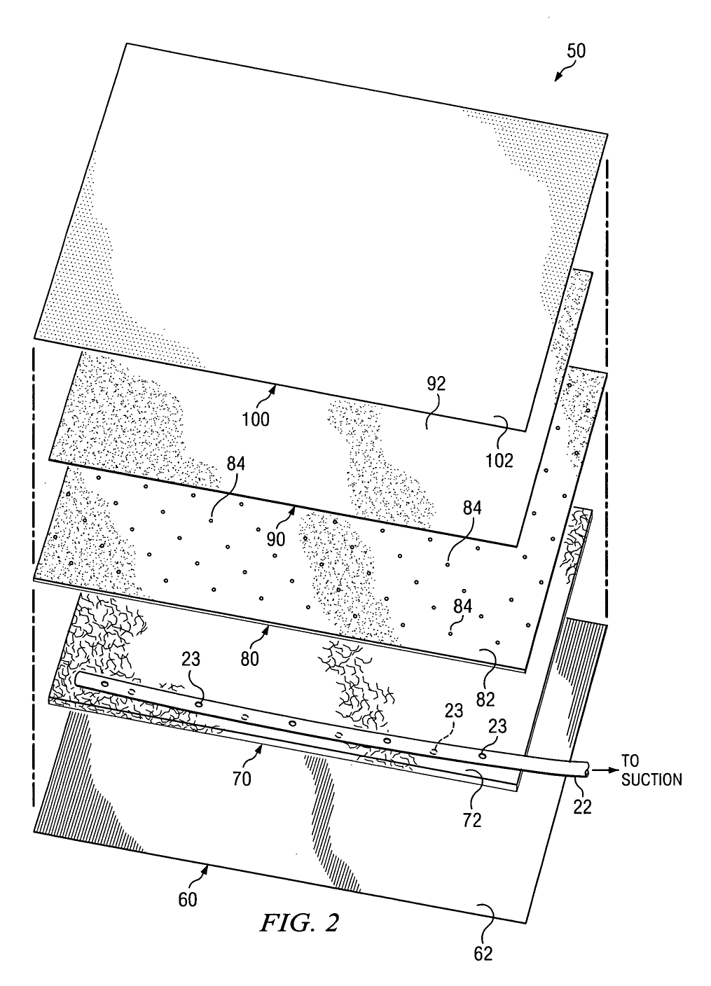

FIGS. 2 and 3 show one example of a fluid collection mat formed from multiple layers of material in accordance with teachings of the present invention. Fluid collection mat 50 may include first layer or base layer 60. Base layer 60 may be formed from fluid impervious material such as light weight polyethylene film or other types of plastic film. The material selected to form base layer 60 is preferably compatible with placing fluid collection mat 50 on a floor, table or other work surface. Base layer 60 includes first surface 61 and second surface 62.

Second layer or fluid pervious layer 70 is preferably disposed on base layer 60. Second layer 70 may be formed from a wide variety of very porous materials such as a porous mesh screen or open cell, porous foam. Second layer 70 may also be formed from filter foam. The selected material preferably allows relatively unrestricted flow of fluids in any direction and relatively unrestricted communication of low pressure suction from conduit 22 in any direction throughout second layer 70. Second layer 70 includes first surface 71 and second surface 72. First surface 71 is preferably disposed on second surface 62 of base layer 60.

Third layer or fluid flow regulator 80 is preferably disposed on second layer 70 opposite from base layer 60. Third layer 80, sometimes referred to as a grid layer, includes first surface 81 and second surface 82. Third layer 80 may be formed from fluid impervious material such as a thin sheet of light weight polyethylene film. Various types of closed cell foams may also be used to form third layer 80.

A relatively small number of holes or fluid flow paths 84 may be formed in grid layer 80. Holes 84 may sometimes be distributed in a relatively uniform pattern or grid across surface 81. The dimensions of each hole 84 may be relatively small to provide relatively small fluid flow area 84a. For some applications the combined total for fluid flow area 284a of holes 84 may be less than twice fluid flow area 22a of conduit 22. See FIG. 3. The total area of first surface 81 is preferably much greater than the combined total for fluid flow areas 84a. For example the total for fluid flow areas 84a may be approximately one percent (1%) or less of the total area of surface 81 depending upon the intended use of an associated fluid collection mat. Holes 84 cooperate with each other to substantially restrict or regulate fluid flow through third layer 80.

Fourth layer or absorbent layer 90 is preferably disposed on third layer 80 opposite from second layer 70. Various types of woven and/or non woven materials may be satisfactorily used to form fourth layer or absorbent layer 90. Absorbent layer 90 may be formed from wood pulp or other similar types of material that absorb substantial amount of fluids, such as water, and distributes the fluid generally uniformly across first surface 91. Fourth layer 90 may also be referred to as a distribution layer. Third layer 80 and absorbent layer 90 cooperate with each other to distribute low pressure suction from conduit 22 generally uniformly throughout second layer 70.

Fifth layer or perforated layer 100 may be disposed on fourth layer 90 opposite from third layer 80. Fifth layer or perforated layer 100 preferably includes a very large number of openings and/or holes which provide substantially unrestricted fluid flow therethrough. Fifth layer or perforated layer 100 may be formed from perforated polyethylene film, non woven materials and/or other porous fabrics or films which allow substantially unrestricted fluid flow from first surface 101 to absorbent layer 90.

Fifth layer 100 may be used to protect absorbent layer 90 from damage or wear. For example fifth layer 100 may be formed from materials which prevent damage to layer 90 from people walking or standing on mat 50. For some applications top layer or perforated layer 100 may be formed as part of absorbent layer 90. Also, for some applications absorbent layer 90 may sometimes be formed from relatively strong, durable materials which do not require the use of fifth layer 100.

When fluids such as water or other liquids initially wet only portions of first surface 91, the absorbent material used to form fourth layer 90 will spread out or distribute the fluid within layer 90. The relatively small number of holes 84 in third layer 80 cooperate with absorbent layer 90 to provide generally uniform distribution of low pressure suction from conduit 22. Second layer 70 contains a large number of open spaces to evenly disperse vacuum from conduit 22. After absorbent layer 90 is saturated with a fluid, the liquid will be drawn through holes 84 in third layer 80. Open spaces or large fluid flow paths associated with second layer 70 allow substantially unrestricted transfer of liquid from third layer 80 to conduit 22.

A fluid collection mat formed in accordance with teachings of the present invention may have a wide variety of configurations and dimensions. For embodiments shown in FIGS. 1-5, fluid collection mat 50 may be described as having a generally rectangular cross section. For other applications a fluid collection mat may be formed in accordance with teachings of the present invention having a circular, oval, square or any other desired configuration.

The thickness and other dimensions associated with the various layers of material used to form fluid collection mat 50 may be substantially varied depending upon each specific application. For example second layer 70 may be formed from relatively thick, sturdy porous material when people may be walking or standing on the associated fluid collection mat 50. For other applications second layer 70 may be formed from a relatively thin layer of porous material when fluid collection mat 70 is placed on a table or other working area. The dimensions associated with each layer of material may be varied to optimize fluid collection capability, cost of manufacture, handling, storage and disposal of the associated mat.

For some applications a fluid barrier may be formed with each layer of material proximate the perimeter of fluid collection mat 50. At least a portion of conduit 22 may extend through the perimeter of mat 50 with one or more fluid barriers (not expressly shown) formed between the exterior of conduit 22 and perimeter 52 of mat 50. Heat sealing techniques and other bonding techniques may be used to form the desired fluid barriers. For embodiments such as shown in FIG. 3, fluid barrier 54 may be formed by heat sealing adjacent portions of base layer 60, third layer 80, and fifth layer 100 with each other proximate perimeter 50 of mat 50. Sonic welding, adhesive bonding and/or RF energy may also be used to form fluid barrier 54.

For some applications such as shown in FIG. 2 portions of conduit 22 may extend along substantially one edge of fluid collection mat 50. A plurality of holes or perforations 23 may be formed within portions of conduit 22 disposed within fluid collection mat 50. For other applications a relatively small portion of conduit 22 may be disposed within mat 50 adjacent to second layer 70 (not expressly shown). Also various types of hose fittings and/or pipe connections (not expressly shown) may be disposed within mat 50 to allow communication between second layer 70 and the exterior of mat 50. Various types of fluid seals and barriers may be formed between portions of a conduit, hose fitting or pipe connection and adjacent portions of perimeter 52 of mat 50. For example a hose connection may be provided at only one corner of fluid collection mat 50. The associated low pressure suction will generally be distributed uniformly throughout second layer 70 as a result of cooperation between fluid flow regulator 80 and absorbent layer 90.

As the present invention and its advantages have been described in detail it should be understood that various changes, substitutions and modifications may be made without departing from the spirit and scope of the invention as defined in the following claims.

What is claimed is:

1. A fluid collection pad comprising:
a base layer formed from generally fluid impervious material;
a second layer disposed on the base layer;
the second layer formed from fluid pervious material offering very little or no resistance to fluid flow therethrough;
a third layer of material disposed on the second layer opposite from the first layer;
the third layer of material formed from generally fluid impervious material;
a limited number of flow paths formed in and extending through the third layer of material to regulate fluid flow through the third layer of material;
a fourth layer of material disposed on the third layer of material opposite from the second layer of material;
the fourth layer of material formed from fluid absorbent material; and
a fluid barrier formed around a perimeter of the layers of material to prevent undesired fluid flow therefrom.

2. The fluid collection pad of claim 1 further comprising:
a fifth layer of material disposed on the fourth layer of material; and
the fifth layer of material formed from highly porous material.

3. The fluid collection pad of claim 1 further comprising a fluid conduit communicating with at least one portion of the second layer.

4. The fluid collection pad of claim 3 further comprising:
each fluid flow path formed in the third layer having a relatively small area to allow fluid flow therethrough;
the fluid conduit having a fluid flow area; and
the total fluid flow area of the fluid flow paths much less than the twice the fluid flow area of the conduit.

5. The fluid collection pad of claim 1 further comprising each layer of material having a generally rectangular configuration.

6. The fluid collection pad of claim 1 further comprising each layer formed from relatively flexible material operable to allow bending and folding of the fluid collection pad.

7. The fluid collection pad of claim 1 further comprising:
the third layer having a first surface with a first surface area;
each fluid flow path having a relatively small area to allow fluid flow therethrough; and
the combined total fluid flow area of the fluid flow paths much less than the first surface area of the third layer.

8. The fluid collection pad of claim 1 further comprising:
a portion of flexible conduit disposed within a portion of the fluid collection pad; and
a plurality of perforations formed in the portion of the fluid conduit.

9. The fluid collection pad of claim 1 further comprising each layer of material having approximately the same general configuration defined in part by each layer having a perimeter corresponding approximately with the perimeter of the other layers.

10. The fluid collection pad of claim 1 further comprising a perimeter defined in part by a fluid barrier to prevent undesired fluid flow therethrough.

11. A disposable fluid collection mat comprising:
a base layer of material with a second layer disposed on the base layer;
the second layer formed from material offering very little resistance to fluid flow therethrough;
a third layer of material disposed on the second layer opposite from the base layer;
the third layer formed from material having high resistance to fluid flow therethrough;
a limited number of openings formed in and extending through the third layer of material to regulate fluid flow through the third layer of material;
a fourth layer of material disposed on the third layer of material opposite from the second layer of material;
the fourth layer of material formed from generally absorbent material;
each layer of material having a perimeter;
a fluid barrier formed around the perimeter of the layers of material to prevent undesired fluid flow therethrough; and
a fluid coupling extending from at least one portion of the mat.

12. The fluid collection mat of claim 11 further comprising:
a fifth layer of material disposed on the fourth layer of material; and
the fifth layer of material formed from highly porous material.

13. The fluid collection mat of claim 11 further comprising the fifth layer and the fourth layer bonded with each other.

14. The fluid collection mat of claim 11 further comprising the fourth layer formed from relatively strong, durable material.

15. The fluid collection mat of claim 11 further comprising the fourth layer of material and the third layer of material bonded with each other.

16. A fluid collection system comprising:
a fluid collection mat coupled with a vacuum source whereby the vacuum source may apply a suction to at least portions of the mat;
the mat defined in part by a first layer of material, a second layer of material disposed on the first layer, a third layer of material disposed on the second layer opposite from the first layer, and a fourth layer of material disposed on the third layer opposite from the second layer;
each layer of material having a perimeter;
a fluid barrier formed around the perimeter of the layers to prevent undesired fluid flow therethrough;
the second layer formed from fluid pervious material allowing substantially unrestricted fluid flow therethrough;
the third layer formed from material offering high resistance to fluid flow therethrough;
a limited number of fluid flow paths formed in and extending through the third layer of material to regulate fluid flow therethrough; and
the fourth layer formed from fluid absorbent material.

17. The fluid collection system of claim 16 further comprising a conduit communicating with at least one portion of the second layer.

18. The fluid collection system of claim 17 further comprising the conduit communicating with the vacuum source.

19. The fluid collection system of claim 11 further comprising:
a first conduit communicating between the second layer and a container; and
a second conduit communicating between the container and the vacuum source.

20. The fluid collection system of claim 19 further comprising the first conduit and the second conduit formed at least in part from flexible material.

21. A method of collecting fluid comprising:
placing a fluid collection mat at a location proximate the fluid;
applying a suction to a portion of the mat;
communicating the suction with other portions of the mat;
absorbing the fluid in a layer of absorbent material; and
regulating the flow of fluid from the layer of absorbent material to the other portions of the mat communicating with the suction.

22. The method of claim 21 further comprising distributing fluid within the absorbent material.

23. The method of claim 21 further comprising communicating the suction from a conduit extending along one edge of a layer of porous material.

24. The method of claim 21 further comprising communicating the suction proximate one corner of the mat.

25. The method of claim 21 further comprising initially collecting the fluid on a porous layer disposed on the layer of absorbent material.

26. A method of forming a fluid collection pad comprising:
forming a base layer with desired dimensions corresponding with a desired surface area and perimeter for the mat;
forming a second layer from generally fluid pervious material;
placing the second layer of material on the first layer of material;
forming a third layer of material from generally fluid pervious material with a limited number of fluid flow paths extending through the third layer of material to regulate fluid flow through the third layer;
placing the third layer of material on the second layer of material;
forming a fourth layer of material from generally absorbent material;
placing the fourth layer of material on the third layer of material; and
forming a fluid barrier around the perimeter of the mat with a fluid coupling extending therefrom.

27. The method of claim 26 further comprising forming the second, third and fourth layers with approximately the same dimensions and configurations as the first layer.

28. The method of claim 26 further comprising placing a fifth layer of highly porous material on the fourth layer.
